(19) United States
(12) Patent Application Publication
(10) Pub. No.: US 2021/0114870 A1
(43) Pub. Date: Apr. 22, 2021

(54) Thermal gas generator

(71) Applicant: John A. Bognar, Belgrade, MT (US)

(72) Inventor: John A. Bognar, Belgrade, MT (US)

(21) Appl. No.: 16/944,132

(22) Filed: Jul. 30, 2020

Related U.S. Application Data
(60) Provisional application No. 62/924,161, filed on Oct. 21, 2019.

(51) Int. Cl. C01B 3/26; C07C 4/04; C07C 4/06; C08L 23/06; B01J 7/00; C07C 4/22; C06B 27/00

(52) U.S. Cl. CPC C01B 3/26; C07C 4/04; C07C 4/06; C08L 23/06; B01J 7/00; C07C 4/22; C07C 11/04; C08L 2207/062; C08L 2207/066; C01B 2203/0227; C01B 2203/0272; C01B 2203/0277; C06B 27/00

(57) ABSTRACT

Devices for generating a desired gas or mixture of gases by thermally decomposing a polymer, and methods of making and using such devices, are provided. The resulting gas or mixture of gases, or a fraction thereof, can be used for any suitable purpose, including but not limited to use as an inflating or lifting gas. The devices and methods of the disclosure provide greater mass and volumetric efficiency for gas generation and storage relative to conventional gas generation solutions and are safer and simpler than compressed gas cylinders or liquefied gas storage.

### CROSS REFERENCE TO RELATED APPLICATION

The present application claims the benefits of U.S. Provisional Application Ser. No. 62/924,161, filed Oct. 21, 2019, entitled “Thermal ethylene gas generator,” which is incorporated herein by this reference in its entirety.

### FIELD

This disclosure relates generally to thermal gas generators and processes and systems for generating a desired gas, and particularly to processes and systems for generating a desired gas by thermally decomposing a polymer.

### BACKGROUND

In many applications, inflatable articles, i.e. articles that can be inflated with a gas, possess several advantages over rigid structures of the same type. Among these advantages are that an inflatable article can be stored in a small space when not inflated, and that inflatable articles can often achieve the same function as rigid counterparts for a fraction of the mass needed. These advantages are crucial considerations in many embodiments, but are particularly important regarding articles or structures adapted for use on aircraft, on spacecraft, in Earth's atmosphere, and in outer space, given that the cost and complexity of launching such articles and structures aboard aircraft or spacecraft can be highly sensitive to the mass and/or volume of the article or structure prior to use.

Finding appropriate devices, methods, and systems to deliver the gas needed to inflate an inflatable structure can often pose various challenges, however. The gas must be generated and delivered to the inflatable article quickly, often in very large quantities; in some aeronautical and astronautical applications, design specifications may call for the production of hundreds of liters of inflation gases in a matter of minutes or even seconds. To accomplish this by conventional means would typically require a housing or tank having substantial mass and volume, which for the reasons previously discussed is often not feasible aboard aircraft or spacecraft and/or in the atmosphere or space. Other applications may require the production of inflation gases in a remote area where it is impractical or impossible to transport tanks or cylinders of gas or to set up conventional gas generators, and in some cases a single person may be required to physically transport the device or system. In all of these applications, as well as others, it is essential to provide compact, lightweight gas delivery devices and systems.

There is thus a need in the art for devices, methods, and systems for generating and delivering a desired gas, or mixture of gases, quickly and from a very small mass and volume. It is further advantageous for such devices, methods, and systems to generate and deliver the gas quickly and in large quantities, while still being suitable for use in challenging environments (the upper atmosphere, space, rugged or remote terrain, etc.).

### SUMMARY

Embodiments and configurations of the present disclosure can address these and other needs.

Multiple Compartment Method

Aspects of the present disclosure include a device having at least a first compartment containing a heat-generating composition, a second compartment containing a polymer, and a separator in thermal contact with the first and second compartments that can provide a mass- and volume-efficient means of generating a gas, such as ethylene gas, for purposes including the inflation of various inflatable structures. The first and second compartments can separately and individually comprise one of steel, aluminum, ceramic, or other heat-resistant materials alone or in combination. Heat from the heat-generating composition can cause the polymer (e.g., polyethylene, polypropylene, polystyrene, trioxane, polyoxymethylene) to break down into ethylene and other product species. The other product species, and even the ethylene, may be further decomposed by thermal and/or catalytic means to increase the molar gas yield per unit mass or unit volume of the generator.

The heat-generating composition can be a thermite composition, i.e. a mixture of a metal oxide and a metal. The metal may, but need not, be selected from the group consisting essentially of vanadium (V) oxide, iron (III) oxide, iron (II,III) oxide, copper (II) oxide, copper (I) oxide, tin (IV) oxide, titanium dioxide, manganese dioxide, manganese (III) oxide, chromium (III) oxide, cobalt (II) oxide, silicon dioxide, nickel (II) oxide, silver oxide, molybdenum trioxide, lead (II,IV) oxide, bismuth (III) oxide, and combinations thereof, and the metal may, but need not, be selected from the group consisting of aluminum, magnesium, silicon, manganese, an alloy of magnesium and aluminum, and combinations thereof. The thermite composition may, but need not, comprise more than one metal, more than one metal oxide, or both.

The separator can transfer thermal energy generated in the first compartment by reaction of the heat-generating composition to the second compartment. The separator can be comprised of steel or another heat-resistant material. Relative variations in material thermal conductivity may be used to tune the behavior of the generator.

While the disclosure is discussed with reference to first and second compartments separated by a separator, it is to be understood that the disclosure can include multiple first and second compartments and/or multiple separators, depending on the configuration.

At least some of the thermal energy can be transferred to the second compartment, thereby thermally decomposing at least some of the polymer to release a desired gas or mixture of gases. Polyethylene is a suitable precursor to produce ethylene and may be selected from low-density polyethylene (LDPE), high-density polyethylene (HDPE), and mixtures thereof. Other polymers suitable for use in the practice of the present invention include polypropylene, polystyrene, trioxane, and polyoxymethylene.

The device can further include an igniter interconnected with the first compartment. The igniter causes the ignition of the heat-generating composition. The igniter can be initiated by one or more of a spark, thermal energy (such as that from a hot wire), flame, or friction. The igniter may ignite a secondary material that burns hot enough to ignite the thermite.

The present disclosure can provide a process for using such devices. The process can have the steps of: (a) initiating, in a first compartment, ignition of a heat-generating composition comprising metal and a metal oxide to release thermal energy; (b) transferring the released thermal energy from the first compartment to a second compartment containing a polymer; and (c) with the thermal energy transferred to the second compartment, initiating the thermal decomposition of the polymer to release a desired gas or mixture of gases.

The reaction of the thermite composition can generate thermal energy. At least some of the thermal energy generated in the thermal or first compartment by the reaction of the thermite can be transferred to the gas-generating or second compartment through a thermal separator. This separator may be one or both of a metal and a nonmetallic compound. In some applications, the thermal separator is a metal wall.

The process can further include the further thermal decomposition of the released gas to increase the molar yield of product gases (of all types).

In embodiments, the second compartment may further contain one or more catalysts configured to promote the thermal decomposition of the polymer.

In embodiments, the gas generator device may be configured to promote further decomposition of the at least one product gas to a secondary product gas by at least one of thermal decomposition and catalytic decomposition. The at least one product gas may, but need not, comprise ethylene gas and the secondary product gas may, but need not, comprise hydrogen gas.

In embodiments, the gas generator device may be configured to cool the at least one product gas. The gas generator device may, but need not, further comprise one or more cooling compartments in fluid communication with the second compartment, configured to receive the at least one product gas from the second compartment and cool the at least one product gas.

In embodiments, the gas generator device may further comprise a component configured to remove or separate a selected species from the at least one product gas. The component may, but need not, be selected from the group consisting of a condenser, a filter, a sieve, and a trap.

In some configurations, the thermal and gas-generating compartments may be stacked with one atop the other. In other configurations, the thermal and gas-generating compartments may be arranged with one partly or completely encased in the other. As will be appreciated, other configurations are envisioned by this disclosure. Channels for gas transport may be integrated by physical structures within or adjacent to the gas-generating material.

Single Compartment Method

In other configurations, the thermal and gas-generating materials may be co-located in a common compartment. For example, the thermal and gas-generating materials may each be in the form of particulates that are mixed homogeneously or heterogeneously together. Alternatively, at least one may be in a rigid form with voids in or among the rigid forms filled by the other. Alternatively, they may be physically separated by a separator or a plurality of separators while sharing a common compartment.

The present disclosure can provide several advantages depending on the particular configuration. The disclosure can provide methods and systems that can generate the desired gas or mixture of gases quickly and can fit in a small volume. The system can therefore be small and lightweight. The gas-generating systems and methods are therefore highly beneficial for rapidly filling inflatable articles, such as meteorological balloons and hypersonic inflatable aerodynamic decelerators (HIADs) for spacecraft.

In embodiments, the at least one product gas may comprise at least one of ethylene gas and hydrogen gas.

In embodiments, the gas generator device may further comprise an igniter interconnected with the first compartment, configured to induce the reaction in the heat-generating composition upon initiation by one or more of a spark, heat, flame, and friction.

In embodiments, at least a portion of the polymer may be surrounded by a heat-resistant material. The at least a portion of the polymer may, but need not, be provided as a lining of a tube made of the heat-resistant material. The at least a portion of the polymer may, but need not, be provided as a pellet having a coating of the heat-resistant material. In other configurations, the gas-generating material may be located inside of a more heat-resistant material, such that (for example) a polymer-lined tube or a coated polymer pellet results.

In aspects of the present disclosure, a gas generator device configured to release at least one product gas by thermal decomposition of a polymer comprises a compartment, containing a heat-generating composition and the polymer; and an igniter interconnected with the compartment, configured to induce a reaction in the heat-generating composition upon initiation by one or more of a spark, heat, flame, and friction, wherein at least one of the following is true: (i) the heat-generating composition and the polymer are physically mixed together: (ii) the heat-generating composition and the polymer are in direct physical contact; (iii) the heat-generating composition and the polymer are spatially arranged proximate to each other to facilitate transfer of thermal energy generated by the reaction of the heat-generating composition to the polymer; (iv) the heat-generating composition is provided as a shaped or molded article comprising voids and the polymer occupies at least a portion of the voids; and (v) the polymer is provided as a shaped or molded article comprising voids and the heat-generating composition occupies at least a portion of the voids.

In embodiments, the heat-generating composition may be a thermite composition, comprising a metal and a metal oxide. The thermite mixture may be any pair of metal and metal oxide species that react according to the thermite (or Goldschmidt) reaction (a complete list is found in Fischer and Grubelich, 1996). The metal may, but need not, be selected from the group consisting essentially of vanadium (V) oxide, iron (III) oxide, iron (II,III) oxide, copper (II) oxide, copper (I) oxide, tin (IV) oxide, titanium dioxide, manganese dioxide, manganese (III) oxide, chromium (III) oxide, cobalt (II) oxide, silicon dioxide, nickel (II) oxide, silver oxide, molybdenum trioxide, lead (II,IV) oxide, bismuth (III) oxide, and combinations thereof, and the metal may, but need not, be selected from the group consisting of aluminum, magnesium, silicon, manganese, an alloy of magnesium and aluminum, and combinations thereof. The thermite composition may, but need not, comprise more than one metal, more than one metal oxide, or both.

The thermite mixture may include more than one metal species. The thermite mixture may include more than one metal oxide species. The thermite mixture may contain other additives that confer advantageous properties.

While a thermite composition is provided as an example, it is to be understood that other heat generating mixtures, compositions, and techniques can be used.

In embodiments, the polymer may comprise at least one of high-density polyethylene, low-density polyethylene, polypropylene, polystyrene, trioxane, and polyoxymethylene.

In embodiments, the gas generator device may further comprise at least one gas transport channel in fluid communication with the compartment, configured to direct flow of the product gas.

In embodiments, at least a portion of the polymer may be surrounded by a heat-resistant material. The at least a portion of the polymer may, but need not, be provided as a lining of a tube made of the heat-resistant material. The at least a portion of the polymer may, but need not, be provided as a pellet having a coating of the heat-resistant material.

In embodiments, the compartment may further contain a catalyst configured to promote the thermal decomposition of the polymer. The product gas mixture may be further decomposed by thermal means.

In embodiments, the thermal decomposition of the polymer may release at least two product gases, and the compartment may further contain a catalyst configured to control kinetics of the thermal decomposition of the polymer to provide a desired production ratio between two or more of the at least two product gases.

In embodiments, the gas generator device may further comprise a second compartment in fluid communication with the compartment, configured to receive the at least one product gas from the compartment and containing a catalyst configured to promote catalytic reforming of the at least one product gas.

In embodiments, the gas generator device may be configured to promote further decomposition of the at least one product gas to a secondary product gas by at least one of thermal decomposition and catalytic decomposition. The at least one product gas may, but need not, comprise ethylene gas and the secondary product gas may, but need not, comprise hydrogen gas.

In embodiments, the gas generator device may be configured to cool the at least one product gas. The gas generator device may, but need not, further comprise a (separate or integral) cooling compartment in fluid communication with the compartment, configured to receive the at least one product gas from the compartment and cool the at least one product gas.

In embodiments, the gas generator device may further comprise a component configured to remove or separate a selected species from the at least one product gas. The component may, but need not, be selected from the group consisting of a condenser, a filter, a sieve, and a trap.

In aspects of the present disclosure, a method for generating a product gas comprises initiating reaction of a heat-generating composition to release thermal energy; transferring at least some of the released thermal energy to a polymer; and decomposing, with the thermal energy transferred to the polymer, at least some of the polymer to release the product gas.

In embodiments, the initiating step may comprise contacting the heat-generating composition with an igniter initiated by one or more of a spark, heat, flame, and friction.

### Advantages

The devices and methods of the present disclosure can have several advantages. One possible advantage of the devices and methods of the present disclosure is that they can generate large quantities of thermal energy, and therefore large quantities of the desired gas or mixture of gases, per unit mass of gas generator. Thus, the devices provided herein can be substantially more compact than conventional devices for generating gases and may therefore allow for the provision of one or more product gases in applications where the significant volume of conventional gas storage solutions (e.g. pressurized cylinders) cannot be accommodated. Additionally, because the heat-generating composition undergoes a reaction that preferably produces little or no offgas—or, in other words, because most of the heat generated by the heat-generating composition is retained in the solid or liquid reaction products—a greater fraction of the thermal energy produced is available to decompose the polymer.

Another possible advantage of the devices and methods of the present disclosure is that they avoid the safety hazards posed by some conventional devices and methods for providing a desired gas. Particularly, pressurized vessels, e.g. gas cylinders, pose various dangers, particularly in challenging environments such airborne and space environments. In the practice of the present disclosure, none of the reactants (i.e. the heat-generating composition), the gas starting material (i.e. the polymer), or the decomposition product (i.e. the product gas) need ever be pressurized, avoiding the dangers posed by pressurized vessels.

Another possible advantage of the devices and methods of the present disclosure is that the starting materials are resistant to phase change and other unwanted physical and chemical changes prior to reaction of the heat-generating composition. By way of non-limiting example, liquid or gas starting materials may be susceptible to undesirable or even dangerous condensation or freezing when employed in low-temperature environments, e.g. the upper atmosphere and space. By remaining in the solid state and generally nonreactive until ignited, the starting materials used in embodiments of the present disclosure avoid this concern and eliminate the need for costly and/or mass- or volume-intensive liquid or gas storage and handling equipment; in terms of simplicity, long-term storage stability, and cost, storage of solid-state materials is generally far more feasible for many applications than dewars or similar devices for storing liquefied gases.

Another possible advantage of the devices and methods of the present disclosure is that the heat-generating composition and the polymer to be decomposed may be provided in separate compartments, or in a simplified reactor comprising a single compartment in which the heat-generating composition and the polymer may be placed in physical contact or close physical proximity, as a particular application may dictate. This versatility in construction allows for use in a still wider range of applications and settings.

Another possible advantage of the devices and methods of the present disclosure is that the heat-generating composition may be ignited, and thus the decomposition of the polymer into the gas(es) of interest, may be ignited by any of several simple and easy methods. Such methods include, but are not limited to, heat, spark, flame, friction, and other pyrotechnic initiation mechanisms.

Another possible advantage of the devices and methods of the present disclosure is that the chemical makeup of the heat-generating composition may be selected or tuned to provide for a desired reaction rate, reaction temperature, amount of thermal energy produced, etc. Particularly, the temperatures at which various widely available polymers decompose are often well-known; as such, the heat-generating composition may be selected (e.g. a particular metal and a metal oxide may be selected as part of a thermite composition for use as a heat-generating composition) to provide an amount of thermal energy sufficient to heat a selected polymer at least to its decomposition temperature. In some embodiments, decomposition of the polymer(s) may produce two or more product gases in a proportion that is at least partially temperature-dependent, and/or it may be desirable to further heat the product gases to trigger a secondary decomposition reaction; by way of non-limiting example, it may be desirable, in some applications, to cause at least some of an ethylene product gas (resulting, e.g., from the thermal decomposition of polyethylene) to be secondarily decomposed to hydrogen gas. As an additional non-limiting example, a higher reaction temperature of the heat-generating composition will in turn increase the amount of thermal energy available to decompose the polymer, which in embodiments may cause the polymer to decompose more rapidly and thus limit the formation of undesirable byproducts, impurities, or offgases. In this way, by selecting an appropriate chemical makeup of the heat-generating composition, it is possible for those skilled in the art to control or tune the amount, composition, formation rate, etc. of the product gas(es).

Another possible advantage of the devices and methods of the present disclosure is that they can produce product gases without the use of a catalyst. Specifically, the very high temperatures generated by the heat-generating compositions, e.g. thermite compositions, of the present disclosure can facilitate “brute force” thermal decomposition without the need for a catalyst, and the paths by which the polymer decomposes at such temperatures can thermodynamically favor the end product gas(es) rather than any intermediate byproducts or impurities. Of course, it may in some embodiments be desirable to include a catalyst and/or to generate a mixture of two or more product gases; such embodiments are expressly contemplated and within the scope of the present disclosure.

These and other advantages will be apparent from the disclosure of the aspects, embodiments, and configurations contained herein.

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together. When each one of A, B, and C in the above expressions refers to an element, such as X, Y, and Z, or class of elements, such as X₁-Xₙ, Y₁-Yₘ, and Z₁-Zₒ, the phrase is intended to refer to a single element selected from X, Y, and Z, a combination of elements selected from the same class (e.g., X₁ and X₂) as well as a combination of elements selected from two or more classes (e.g., Y₁ and Zₒ).

It is to be noted that the term “a” or “an” entity refers to one or more of that entity. As such, the terms “a” (or “an”), “one or more” and “at least one” can be used interchangeably herein. It is also to be noted that the terms “comprising,” “including,” and “having” can be used interchangeably.

The term “means” as used herein shall be given its broadest possible interpretation in accordance with 35 U.S.C., Section 112(f) and/or Section 112, Paragraph 6. Accordingly, a claim incorporating the term “means” shall cover all structures, materials, or acts set forth herein, and all of the equivalents thereof. Further, the structures, materials or acts and the equivalents thereof shall include all those described in the summary of the disclosure, brief description of the drawings, detailed description, abstract, and claims themselves.

Polyethylene is a polymer comprising nonpolar, saturated, high molecular weight hydrocarbons. Polyethylenes are divided mainly into two types. (1) low density polyethylene, and (2) high density polyethylene. Polyethylene can also be classified as ultra-high-molecular-weight polyethylene (UHMWPE), ultra-low-molecular-weight polyethylene (ULMWPE), high-molecular-weight polyethylene (HMWPE), high-density cross-linked polyethylene (HDXLPE), cross-linked polyethylene (PEX or XLPE), medium-density polyethylene (MDPE), linear low-density polyethylene (LLDPE), and very-low-density polyethylene (VLDPE).

The term “thermite” refers to a mixture of a metal fuel and a metal oxide oxidizer. The metal may, but need not, be selected from the group consisting essentially of vanadium (V) oxide, iron (III) oxide, iron (II,III) oxide, copper (II) oxide, copper (I) oxide, tin (IV) oxide, titanium dioxide, manganese dioxide, manganese (III) oxide, chromium (III) oxide, cobalt (II) oxide, silicon dioxide, nickel (II) oxide, silver oxide, molybdenum trioxide, lead (II,IV) oxide, bismuth (III) oxide, and combinations thereof, and the metal may, but need not, be selected from the group consisting of aluminum, magnesium, silicon, manganese, an alloy of magnesium and aluminum, and combinations thereof. The thermite composition may, but need not, comprise more than one metal, more than one metal oxide, or both.

When ignited by heat, thermite undergoes an exothermic reduction-oxidation (redox) reaction.

Unless otherwise noted, all component or composition levels are in reference to the active portion of that component or composition and are exclusive of impurities, for example, residual solvents or by-products, which may be present in commercially available sources of such components or compositions.

All percentages and ratios are calculated by total composition weight, unless indicated otherwise.

Every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein. By way of example, the phrase from about 2 to about 4 includes the whole number and/or integer ranges from about 2 to about 3, from about 3 to about 4 and each possible range based on real (e.g., irrational and/or rational) numbers, such as from about 2.1 to about 4.9, from about 2.1 to about 3.4, and so on.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 depicts a device according to some embodiments of the present disclosure;

FIG. 2 depicts a process according to some embodiments of the present disclosure;

FIG. 3 depicts another device for generating a desired gas or mixture of gases according to some embodiments of the present disclosure;

FIG. 4 depicts another device for generating a desired gas or mixture of gases according to some embodiments of the present disclosure;

FIG. 5 depicts yet another device for generating a desired gas or mixture of gases according to some embodiments of the present disclosure;

FIG. 6 depicts another process according to some embodiments of the present disclosure; and

FIG. 7 depicts another process according to some embodiments of the present disclosure.

### DETAILED DESCRIPTION

Devices for generating a desired gas or mixture of gases, and methods for making and using such devices, are provided herein. Compared to conventional devices and systems for gas generation, the devices and systems of the present disclosure may have any one or more of several advantages and benefits, including but not limited to decreased mass, decreased volume or spatial footprint, an ability to generate the desired gas or mixture of gases in greater quantities, and an ability to generate the desired gas or mixture of gases more rapidly.

In some configurations, a first volume can be filled with a thermite mixture or other heat-generating mixture; such mixture is commonly not gas generating on its own (or is substantially free of gaseous byproduct(s) during release of thermal energy). A second volume can be filled with a gas-generating polymeric composition, such as any one or more of polyethylene, polypropylene, polystyrene, trioxane, and polyoxymethylene, which may be in forms such as but not limited to pellets, sheets, tubes, rods, fibers, or custom molded shapes. The two volumes are commonly in thermal contact with each other, advantageously through a medium that can moderate or control the rate of heat transfer, though such control is not required and is not used in every embodiment.

To start the generator, the heat-generating material, e.g., thermite mixture (such as but not limited to a mixture of aluminum metal and iron (III) oxide), can be ignited to produce heat. As heat or thermal energy is conducted from the first volume to the second volume, a mixture of gases including, in the case of polyethylene, a substantial portion of ethylene is initially produced as the polyethylene decomposes. This gas mixture may be used as-is, thermally and/or catalytically treated to yield a more desirable gas mixture, and/or have undesirable components removed through means such as but not limited to filters, sieves, traps, or condensers.

In some configurations, a common volume can be filled with both 1) a thermite mixture or other heat-generating mixture (such mixture is preferably not gas generating on its own), and 2) a gas-generating polymeric composition, such as any one or more of polyethylene, polypropylene, polystyrene, trioxane, and polyoxymethylene, which may be in forms such as but not limited to pellets, sheets, tubes, rods, fibers, or custom molded shapes. The two species can be in thermal contact with each other by virtue of either 1) being a physical mixture, or 2) other direct physical contact, or 3) physical proximity, or 4) being a physical construct in which either the thermite, the polymer, or both are molded shapes, and the one species occupies at least a portion of the voids in the other.

To start the generator, the thermal-generating material, e.g., thermite mixture (such as but not limited to a mixture of aluminum metal and iron (III) oxide), can be ignited to produce a hot slag. The polyethylene or other polymeric material which is now in direct contact with the slag will thermally decompose yielding a mixture of gases including, in the case of polyethylene, a substantial portion of ethylene. This gas mixture may be used as-is, thermally and/or catalytically treated to yield a more desirable gas mixture, and/or have undesirable components removed through means such as but not limited to filters, sieves, traps, or condensers.

Various embodiments of the gas generator device will now be discussed with reference to the figures.

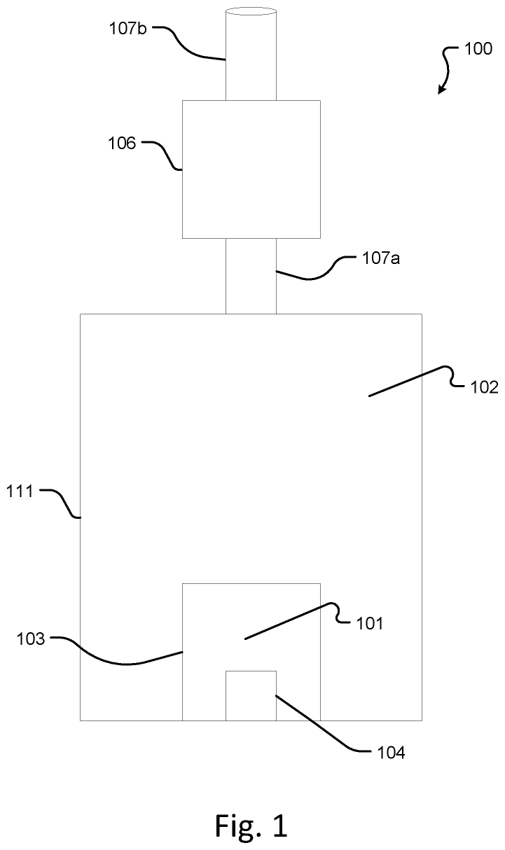

FIG. 1 depicts a non-limiting configuration of a gas generator device 100. The gas generator device 100 comprises a first compartment 101 containing a heat-generating composition and a second compartment 102 containing a polymer. The first 101 and second 102 compartments are typically separated from an outside environment by a wall 111 and from each other by a separator 103. The separator 103 is in thermal contact with the first 101 and second 102 compartments. Thermal energy generated in the first compartment 101 by reaction of the heat-generating composition is transferred to the second compartment 102 by the separator 103, whereby at least some of the thermal energy transferred to the second compartment 102 thermally decomposes at least some of the polymer to release at least one product gas.

The first 101 and second 102 compartments have first and second compartment volumes, respectively. The gas generator device 100 has a device volume. In some configurations the device volume can be the sum of the first 101 and second 102 compartment volumes. In some configurations, the device volume can be more than the sum of the first 101 and second 102 compartment volumes. In some configurations, the first 101 and second 102 compartments can be stacked one atop the other; it will be appreciated that the compartments can be stacked in any order. In other configurations, the first 101 and second 102 compartments can be arranged with one of the compartments partly or completely encased in the other, as for example depicted without limitation in FIG. 1. One or both of first 101 and second 102 compartments may be comprised of, separately and independently, one or more of steel, aluminum, and ceramic.

In some embodiments, the first compartment 101 is configured with one or more vents (not depicted).

Most typically, the heat-generating composition comprises a thermite composition, which in turn comprises a metal (i.e. a fuel) and a metal oxide (i.e. an oxidizer). The thermite reaction, i.e. the exothermic reduction-oxidation reaction between a metal fuel and a metal oxide when ignited by heat, has been known for well over a century; see, e.g., U.S. Pat. No. 906,009, entitled “Manufacture of thermic mixtures,” issued 8 Dec. 1908 to Goldschmidt (“Goldschmidt”), the entirety of which is incorporated herein by reference. The thermite reaction is generally non-explosive but can create intense heat and high temperatures; it thus finds a variety of useful applications, (e.g. welding, metal refining, disabling munitions, incendiary weapons, and pyrotechnic initiators) and so is widely, and (for many formulations) inexpensively, available from many suppliers. The metal may, but need not, be selected from the group consisting essentially of vanadium (V) oxide, iron (III) oxide, iron (II,III) oxide, copper (II) oxide, copper (I) oxide, tin (IV) oxide, titanium dioxide, manganese dioxide, manganese (III) oxide, chromium (III) oxide, cobalt (II) oxide, silicon dioxide, nickel (II) oxide, silver oxide, molybdenum trioxide, lead (II,IV) oxide, bismuth (III) oxide, and combinations thereof, and the metal may, but need not, be selected from the group consisting of aluminum, magnesium, silicon, manganese, an alloy of magnesium and aluminum, and combinations thereof. The thermite composition may, but need not, comprise more than one metal, more than one metal oxide, or both.

In one configuration, the heat-generating composition comprises a thermite composition that comprises a mixture of ferric oxide and aluminum. The chemical reaction of this thermite mixture is shown below in chemical equation (1):

Fe₂O₃(s)+2Al(s)→2Fe(s)+Al₂O₃(s) (1)

The thermite chemical reaction is exothermic and releases a large quantity of thermal energy, resulting in temperatures sufficient to produce an aluminum oxide slag and molten iron. The enthalpy or heat of reaction (ΔH° value) for the thermite reaction is about −849 k (e.g., −849 kJ per mole Fe₂O₃). The thermite reaction does not require external oxygen and can, therefore, proceed in locations with limited or no air flow (e.g. in space), or even under water. Furthermore, the reaction of many types and mixtures of thermite does not produce any gases which might carry away some of the heat of the reaction or produce an explosive excess of pressure.

It can be appreciated that the heat-generating composition can generate very large amounts of thermal energy per unit mass of the heat-generating composition. A compact gas generating system can thus be achieved by producing such large amounts of thermal energy per unit mass of the heat-generating composition. Furthermore, in many embodiments, substantially most of the heat generated remains available to decompose the polymer because gaseous byproducts are not produced; that is, most of the heat is retained in the liquid and/or solid reaction products as a source of thermal energy.

Typically, at least some of the thermal energy transferred to the second compartment 102 by the separator 103 thermally decomposes some of the polymer contained in the second compartment 102. The thermal decomposition of the polymer releases one or more product gases. By way of non-limiting example, polyethylene can be thermally decomposed to ethylene gas, and in some embodiments at least a portion of the ethylene gas may be secondarily decomposed (either thermally or catalytically) to hydrogen gas.

In some embodiments, at least about 99 mole % of the polymer may be converted to the one or more product gases. More generally, at least 95 mole %, even more generally at least about 90 mole %, yet even more generally at least about 80 mole %, still yet even more generally at least about 70 mole %, still yet even more generally at least about 60 mole %, still yet even more generally at least about 50 mole %, still yet even more generally at least about 40 mole %, still yet even more generally at least about 30 mole %, still yet even more generally at least about 20 mole %, or yet still even more generally at least about 10 mole % of the polymer may be converted to the one or more product gases.

It can be appreciated that, in many embodiments, there is no need to control one or both of the temperature or thermal energy transfer within the device 100. As a result, the device 100 can be configured to transfer thermal energy rapidly between the first 101 and second 102 compartments, thereby decomposing the polymer to release the one or more product gases more rapidly than current gas generation systems. Moreover, the device 100 can be more easily constructed and operated than other gas generators; for example, there is not always a need to have the polymer decomposition occur at any specific temperature, so neither the reaction of the heat-generating composition nor the transfer of thermal energy from the first 101 to the second 102 compartment must necessarily be regulated. This contrasts with catalytic decomposition methods, which require the catalyst to be operated at specific temperatures, pressures, and reactant flow rates. Even more advantageously, in those embodiments where control over one or both of the temperature or the rate of energy transfer within the device 100 is required or desired, such control can be achieved by varying the chemical makeup of the thermite or other heat-generating composition within the first compartment 101, without the need to rebuild or retrofit the device 100 itself.

The gas generator device 100 may further include an igniter 104 interconnected with the first compartment. The igniter 104 causes the ignition of the heat-generating composition. In some configurations, a spark generated within the igniter 104 initiates the ignition process. In other configurations, the ignition process is initiated by thermal energy generated within the igniter 104. The thermal energy provided within igniter 104 may be from a hot wire. In other configurations, the initiating energy within igniter 104 may be from flame. In other configurations, the initiating energy within the igniter 104 may be provided by friction.

The igniter 104 may further comprise an ignition aperture in the first compartment 101. The ignition aperture may be configured with a safety-delay switch system.

The gas generator device 100 may further include a heat exchanger 106 interconnected with the second compartment 102. The heat exchanger 106 is configured to cool the product gas(es) released from the polymer. In accordance with some embodiments, the heat exchanger 106 may be interconnected to outlet 107a of the second compartment 102. The exchanger 106 cools the product gas(es) exiting the second compartment 102 through outlet 107a and releases the cooled gas through outlet 107b.

It is to be expressly understood that that the first 101 and second 102 compartments can be spatially arranged in any suitable configuration. By way of non-limiting example, in some embodiments, the compartments can be stacked atop each other, while in other embodiments one of the compartments can be partially or completely encased within or surrounded by the other compartment.

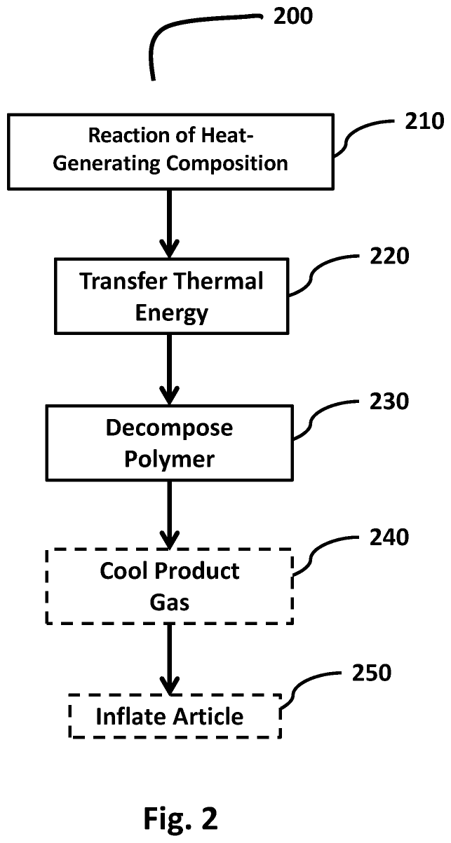

FIG. 2 depicts a process 200 for using the gas generator device 100 of FIG. 1.

In step 210, reaction of a heat-generating composition is initiated in a first compartment 101. The reaction releases thermal energy. The heat-generating composition may be a thermite composition comprised of a metal and a metal oxide. The metal may, but need not, be selected from the group consisting of vanadium (V) oxide, iron (III) oxide, iron (II,III) oxide, copper (II) oxide, copper (I) oxide, tin (IV) oxide, titanium dioxide, manganese dioxide, manganese (III) oxide, chromium (III) oxide, cobalt (II) oxide, silicon dioxide, nickel (II) oxide, silver oxide, molybdenum trioxide, lead (II,IV) oxide, bismuth (III) oxide, and combinations thereof, and the metal may, but need not, be selected from the group consisting of aluminum, magnesium, silicon, manganese, an alloy of magnesium and aluminum, and combinations thereof. The thermite composition may, but need not, comprise more than one metal, more than one metal oxide, or both.

Step 210 may further include contacting the heat-generating composition with an igniter to initiate the reaction. In some configurations the reaction may be initiated by contacting the igniter with one of a hot wire or a spark. In other configurations, flame may initiate the reaction of the heat-generating composition via the igniter. In yet other configurations, friction may initiate reaction of the heat-generating composition via the igniter.

In step 220, the energy released by the reaction of the heat-generating composition is transferred from the first compartment 101 to a second compartment 102. A polymer is contained in the second compartment. Non-limiting examples of the polymer include various forms of polyethylene (e.g. low-density polyethylene (LDPE), high-density polyethylene (HDPE), and mixtures thereof), polypropylene, polystyrene, trioxane, and polyoxymethylene.

In step 230, the thermal energy transferred to the second compartment 102 decomposes the polymer to release one or more product gases. Step 230 may further include transferring the released thermal energy from the first compartment 101 to the second compartment 102 through a separator 103. The material and construction of the separator 103, and the composition and amount of the polymer in the second compartment 102, can be selected to provide for a desired amount or rate of production of the product gas(es).

In optional step 240, the released product gas(es) may be cooled, in some embodiments by a heat exchanger.

In optional step 250, the released gas may be used for one of: inflation of a meteorological balloon; inflation of other types of balloons; inflation of a blimp; inflation of a HIAD; inflation of an inflatable article; pressurization of a gas storage cylinder; and the like.

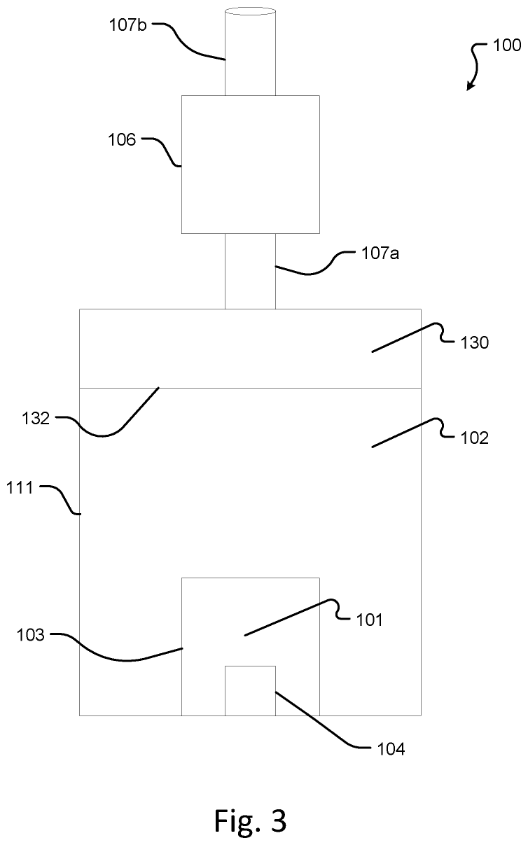

FIG. 3 depicts a device for generating at least one product gas according to various embodiments as described in the above Summary and Detailed Description and hereinbelow. More specifically, FIG. 3 depicts a device 100 having first 101, second 102, and third 130 compartments, with the second compartment 102 positioned between the first compartment 101 and the third compartment 130. The first 101, second 102, and third 130 compartments have walls 111. A separator 103 separates the first 101 and second 102 compartments. The separator 103 is in thermal contact with the first 101 and second 102 compartments. A partition 132 separates the second 102 and third 130 compartments. The first compartment 101 contains a heat-generating composition (not depicted); the second compartment 102 contains a polymer (not depicted); and the third compartment 130 contains a catalyst which can reform the gas exiting compartment 102.

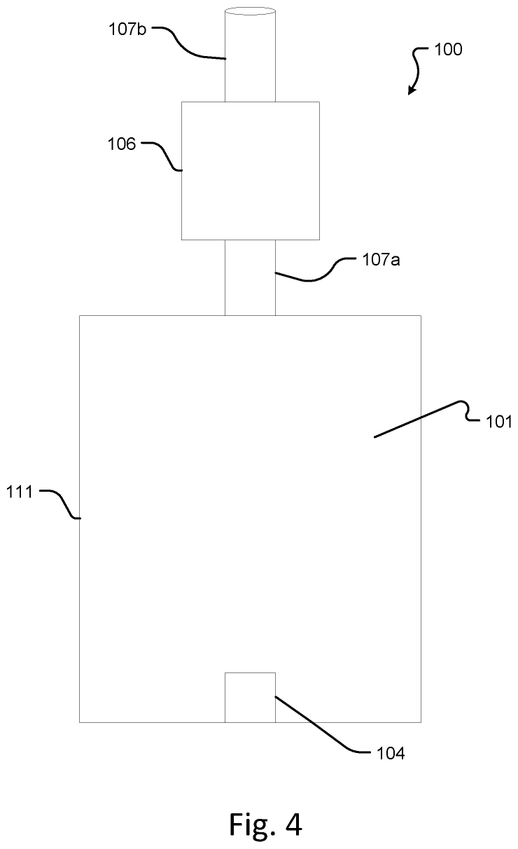

FIG. 4 depicts a non-limiting configuration of a gas generator device 100. The gas generator device 100 comprises a single compartment 101 containing both the heat-generating composition and the polymer; in other words, the polymer is provided in the same compartment, and optionally mixed together with or otherwise in physical contact with, the heat-generating composition, in contrast to the device 100 depicted in FIG. 1. The compartment 101 is typically separated from an outside environment by a wall 111. Thermal energy generated by reaction of the heat-generating composition can be received by the polymer, whereby at least some of the thermal energy thermally decomposes at least some of the polymer to release at least one product gas.

In accordance with some embodiments, the compartment 101 may be defined by a wall 111.

Typically, at least some of the thermal energy available to the polymer due to the reaction of the heat-generating composition in the compartment 101 thermally decomposes some of the polymer contained in the compartment 101. The thermal decomposition of the polymer releases one or more product gases. By way of non-limiting example, polyethylene can be thermally decomposed to ethylene gas, and in some embodiments at least a portion of the ethylene gas may be secondarily decomposed (either thermally or catalytically) to hydrogen gas.

In some embodiments, at least about 99 mole % of the polymer may be converted to the one or more product gases. More generally, at least 95 mole %, even more generally at least about 90 mole %, yet even more generally at least about 80 mole %, still yet even more generally at least about 70 mole %, still yet even more generally at least about 60 mole %, still yet even more generally at least about 50 mole %, still yet even more generally at least about 40 mole %, still yet even more generally at least about 30 mole %, still yet even more generally at least about 20 mole %, or yet still even more generally at least about 10 mole % of the polymer may be converted to the one or more product gases.

It can be appreciated that, in many embodiments, there is no need to control one or both of the temperature or thermal energy transfer within the device 100. Moreover, the device 100 can be more easily constructed and operated than other gas generators; for example, the absence of a second compartment may simplify the design of the device 100 and be suitable for applications in which transfer of substantially all of the thermal energy generated by reaction of the heat-generating composition to the polymer is desirable. Even more advantageously, in those embodiments where control over one or both of the temperature or the rate of energy transfer within the device 100 is required or desired, such control can be achieved by varying the chemical makeup of the thermite or other heat-generating composition within the compartment 101, and/or the spatial arrangement of the polymer relative to the heat-generating composition in the compartment 101, without the need to redesign the device 100 itself.

The gas generator device 100 may further include an igniter 104 interconnected with the compartment 101. The igniter 104 causes the ignition of the heat-generating composition. In some configurations, a spark generated within the igniter 104 initiates the ignition process. In other configurations, the ignition process is initiated by thermal energy generated within the igniter 104. The thermal energy provided within igniter 104 may be from a hot wire. In other configurations, the initiating energy within igniter 104 may be from flame. In other configurations, the initiating energy within the igniter 104 may be provided by friction.

The igniter 104 may further comprise an ignition aperture in the compartment 101. The ignition aperture may be configured with a safety-delay switch system.

The gas generator device 100 may further include a heat exchanger 106 interconnected with the compartment 101. The heat exchanger 106 is configured to cool the product gas(es) released from the polymer. In accordance with some embodiments, the heat exchanger 106 may be interconnected to outlet 107a of the compartment 101. The exchanger 106 cools the product gas(es) exiting the compartment 101 through outlet 107a, with the cooled gas exiting the exchanger 106 via outlet 107b.

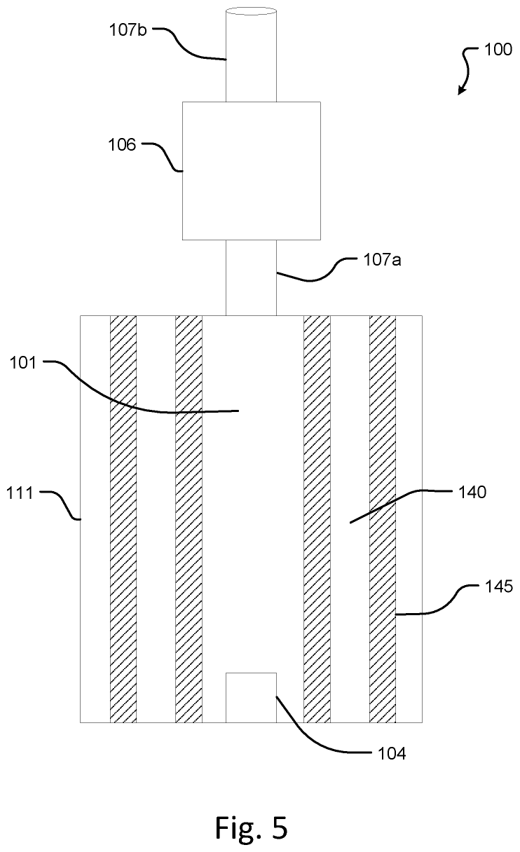

FIG. 5 depicts a non-limiting configuration of a gas generator device 100. The gas generator device 100 comprises a single or common compartment 101 containing both the heat-generating composition and the polymer in discrete form such as rods 140, in contrast to the device 100 depicted in FIG. 1. The compartment 101 is typically separated from an outside environment by a wall 111. Thermal energy generated by reaction of the heat-generating composition can be received by the polymer, whereby at least some of the thermal energy thermally decomposes at least some of the polymer to release at least one product gas. Transfer of the energy generated by the heat-generating composition to the polymer is moderated by a separator layer 145. As will be appreciated, the separator layer 145 which moderates heat transfer between the heat-generating composition and the polymer can be a continuous or discontinuous layer on the polymer, the heat-generating composition, or both depending on the configuration.

Typically, at least some of the thermal energy available to the polymer due to the reaction of the heat-generating composition in the compartment 101 thermally decomposes some of the polymer contained in the compartment 101. The thermal decomposition of the polymer releases one or more product gases. By way of non-limiting example, polyethylene can be thermally decomposed to ethylene gas, and in some embodiments at least a portion of the ethylene gas may be secondarily decomposed (either thermally or catalytically) to hydrogen gas.

In some embodiments, at least about 99 mole % of the polymer may be converted to the one or more product gases. More generally, at least 95 mole %, even more generally at least about 90 mole %, yet even more generally at least about 80 mole %, still yet even more generally at least about 70 mole %, still yet even more generally at least about 60 mole %, still yet even more generally at least about 50 mole %, still yet even more generally at least about 40 mole %, still yet even more generally at least about 30 mole %, still yet even more generally at least about 20 mole %, or yet still even more generally at least about 10 mole % of the polymer may be converted to the one or more product gases.

It can be appreciated that, in many embodiments, there is no need to control one or both of the temperature or thermal energy transfer within the device 100. Moreover, the device 100 can be more easily constructed and operated than other gas generators; for example, the absence of a second compartment may simplify the design of the device 100 and be suitable for applications in which transfer of substantially all of the thermal energy generated by reaction of the heat-generating composition to the polymer is desirable. Even more advantageously, in those embodiments where control over one or both of the temperature or the rate of energy transfer within the device 100 is required or desired, such control can be achieved by varying the chemical makeup of the thermite or other heat-generating composition within the compartment 101, and/or the spatial arrangement of the polymer relative to the heat-generating composition in the compartment 101, without the need to redesign the device 100 itself.

The gas generator device 100 may further include an igniter 104 interconnected with the compartment 101. The igniter 104 causes the ignition of the heat-generating composition. In some configurations, a spark generated within the igniter 104 initiates the ignition process. In other configurations, the ignition process is initiated by thermal energy generated within the igniter 104. The thermal energy provided within igniter 104 may be from a hot wire. In other configurations, the initiating energy within igniter 104 may be from flame. In other configurations, the initiating energy within the igniter 104 may be provided by friction.

The igniter 104 may further comprise an ignition aperture in the compartment 101. The ignition aperture may be configured with a safety-delay switch system.

The gas generator device 100 may further include a heat exchanger 106 interconnected with the compartment 101. The heat exchanger 106 is configured to cool the product gas(es) released from the polymer. In accordance with some embodiments, the heat exchanger 106 may be interconnected to outlet 107a of the compartment 101. The exchanger 106 cools the product gas(es) exiting the compartment 101 through outlet 107a, with the cooled gas exiting the exchanger 106 via outlet 107b.

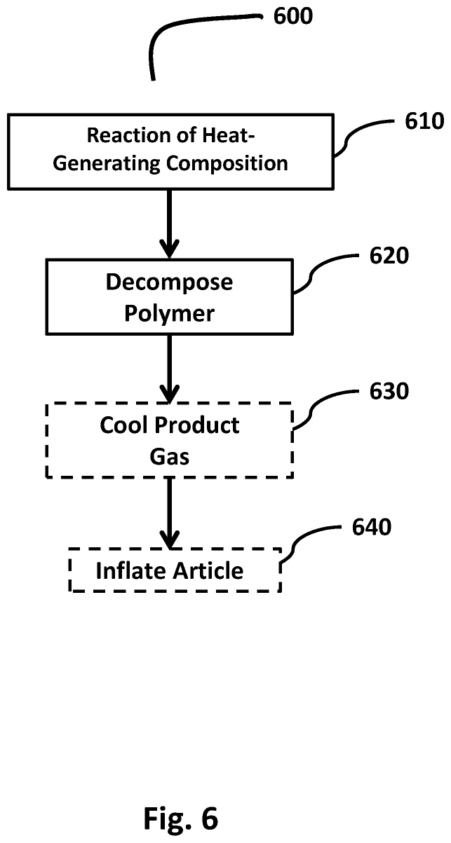

FIG. 6 depicts a process 600 for using the gas generator device 100 of FIGS. 4 and 5.

In step 610, reaction of a heat-generating composition is initiated in a compartment 101. The reaction releases thermal energy. The heat-generating composition may comprise a thermite composition comprising a metal and a metal oxide. The metal may, but need not, be selected from the group consisting of vanadium (V) oxide, iron (III) oxide, iron (II,III) oxide, copper (II) oxide, copper (I) oxide, tin (IV) oxide, titanium dioxide, manganese dioxide, manganese (III) oxide, chromium (III) oxide, cobalt (II) oxide, silicon dioxide, nickel (II) oxide, silver oxide, molybdenum trioxide, lead (II,IV) oxide, bismuth (III) oxide, and combinations thereof, and the metal may, but need not, be selected from the group consisting of aluminum, magnesium, silicon, manganese, an alloy of magnesium and aluminum, and combinations thereof. The thermite composition may, but need not, comprise more than one metal, more than one metal oxide, or both.

Step 610 may further include contacting the heat-generating composition with an igniter to initiate the reaction. In some configurations the reaction may be initiated by contacting the igniter with one of a hot wire or a spark. In other configurations, flame may initiate the reaction of the heat-generating composition via the igniter. In yet other configurations, friction may initiate reaction of the heat-generating composition via the igniter.

In step 620, the thermal energy generated by reaction of the heat-generating composition decomposes a polymer in the compartment 101 to release one or more product gases. In contrast to the device depicted in FIGS. 1A and 1B and the method depicted in FIG. 2, the device depicted in FIG. 5 and the method depicted in FIG. 6 do not include or require transfer of thermal energy to a separate compartment via a separator: rather, because the heat-generating composition and the polymer are provided in the same single compartment 101 (either with or without a separator 145), the thermal energy, or some significant portion thereof, is generally immediately available to decompose the polymer into the one or more product gases.

In optional step 630, the released product gas(es) may be cooled, in some embodiments by a heat exchanger.

In optional step 640, the released gas may be used for one of: inflation of a meteorological balloon; inflation of other types of balloons; inflation of a blimp; inflation of a HIAD; inflation of an inflatable article; pressurization of a gas storage cylinder; and the like.

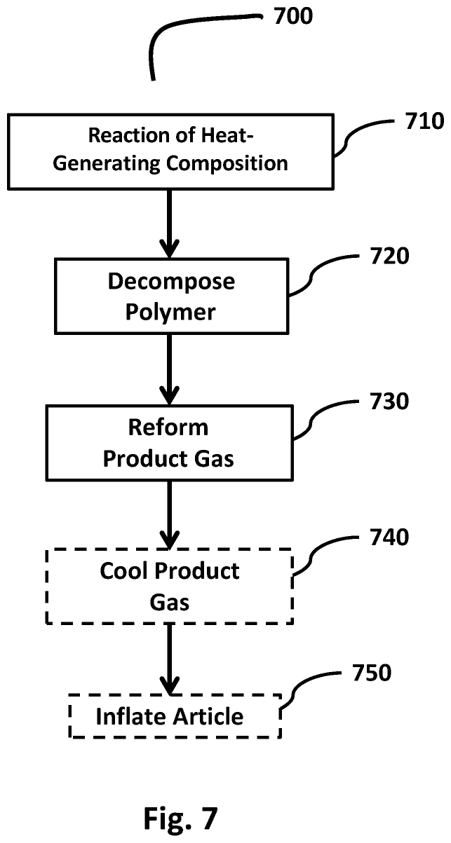

FIG. 7 depicts a process 700 for using a gas generator device according to any one or more of FIGS. 1A, 1B, 4, and 5.

In step 710, reaction of a heat-generating composition is initiated in a compartment. The reaction releases thermal energy. The heat-generating composition may comprise a thermite composition comprising a metal and a metal oxide. The metal may, but need not, be selected from the group consisting of vanadium (V) oxide, iron (III) oxide, iron (II,III) oxide, copper (II) oxide, copper (I) oxide, tin (IV) oxide, titanium dioxide, manganese dioxide, manganese (III) oxide, chromium (III) oxide, cobalt (II) oxide, silicon dioxide, nickel (II) oxide, silver oxide, molybdenum trioxide, lead (II,IV) oxide, bismuth (III) oxide, and combinations thereof, and the metal may, but need not, be selected from the group consisting of aluminum, magnesium, silicon, manganese, an alloy of magnesium and aluminum, and combinations thereof. The thermite composition may, but need not, comprise more than one metal, more than one metal oxide, or both.

Step 710 may further include contacting the heat-generating composition with an igniter to initiate the reaction. In some configurations the reaction may be initiated by contacting the igniter with one of a hot wire or a spark. In other configurations, flame may initiate the reaction of the heat-generating composition via the igniter. In yet other configurations, friction may initiate reaction of the heat-generating composition via the igniter.

In step 720, the thermal energy generated by reaction of the heat-generating composition decomposes a polymer in the compartment to release one or more product gases. Step 720 may in some embodiments include or require transfer of thermal energy to a separate compartment via a separator, as in the device depicted in FIGS. 1A and 1B and the method depicted in FIG. 2, whereas in other embodiments step 720 may omit such transfer, e.g. by providing the heat-generating composition and the polymer in a single compartment as in the device depicted in FIG. 5 and the method depicted in FIG. 6.

In step 730, the one or more product gases are subjected to further chemical processing. Particularly, step 730 may in embodiments include reformation of one or more product gases. In such embodiments, the method 700 may employ a catalyst configured to facilitate catalytic reformation of the one or more product gases. Such catalyst may be provided in any desired spatial arrangement (e.g. a fixed bed), and may be present either in the compartment in which the one or more product gases are formed (i.e. the compartment containing the polymer), or in a separate compartment configured to receive the one or more product gases.

In optional step 740, the released product gas(es) may be cooled, in some embodiments by a heat exchanger.

In optional step 750, the released gas may be used for one of: inflation of a meteorological balloon; inflation of other types of balloons; inflation of a blimp; inflation of a HIAD; inflation of an inflatable article; pressurization of a gas storage cylinder; and the like.

Embodiments of the devices and methods disclosed herein may be directed to the thermal decomposition of any one or more polymers such as polyethylene, polypropylene, polystyrene, trioxane, polyoxymethylene, and combinations and mixtures thereof.

Embodiments of the devices and methods disclosed herein may be directed to the production of any one or more product gases, but particularly may be directed to the production of ethylene gas, and/or (either directly or by secondary thermal or catalytic decomposition of ethylene) hydrogen gas. Ethylene gas, or hydrogen gas, or the combination of ethylene and hydrogen gases may, in embodiments, generally make up at least about 75 mol %, more generally at least about 70 mol %, even more generally at least about 65 mol %, yet even more generally at least about 60 mol %, still yet even more generally at least about 55 mol %, still yet even more generally at least about 50 mol %, still yet even more generally at least about 45 mol %, still yet even more generally at least about 40 mol %, still yet even more generally at least about 35 mol %, still yet even more generally at least about 30 mol %, or still yet even more generally at least about 25 mol % of the total product gas content.

In embodiments of the devices and methods disclosed herein, the composition of the product gas(es) may be such that it is not necessary to provide additional heat or other (or, in some cases, any) energy inputs to maintain most or all of the product gas(es) in the desired gaseous state after formation of the gas. By way of non-limiting example, the product gas(es) may in some embodiments be passively or actively cooled to ambient or near-ambient temperatures (e.g. at least substantially, if not entirely, free of added heat or thermal energy relative to ambient conditions), without risk of undesirable condensation of product gas(es). In this way, the devices and methods disclosed herein may advantageously serve differing purposes relative to gas generation devices and methods of the prior art.

In some embodiments, the precise chemical composition or properties of the one or more product gas(es) are not a consideration, or at least are not as crucial a consideration as the rate or amounts (whether molar or mass amounts) in which the product gas(es) can be generated; by way of non-limiting example, it may be desirable to produce as great a molar quantity of gas as possible to inflate an inflatable article to the greatest extent possible, since volume is directly related not to mass of the gas but to its molar quantity. In these applications, it may be desirable to cause the polymer to decompose in the first instance, and/or to cause one or more product gas(es) to undergo secondary decomposition, into as “small” (in molecular weight terms) a gas as possible to increase the volume of gas produced without requiring additional mass of materials. One such desirable “small” gas is hydrogen gas (H₂). Thus, in embodiments, a heat-generating composition may be provided that provides temperatures great enough to rapidly facilitate decomposition of, e.g., ethylene gas (produced, e.g., by decomposition of polyethylene) to hydrogen gas. In other embodiments, a catalyst may be provided in the compartment containing the polymer that catalyzes the decomposition of a product gas into hydrogen gas or another “small” gas.

In some embodiments, it may be necessary to minimize or eliminate byproducts, impurities, and other undesirable species in the product gas(es). However, limitations on the availability of a suitable polymer may necessitate the use of a polymer that is susceptible to the production of such byproducts, impurities, and undesirable species. By way of non-limiting example, higher hydrocarbons such as C4 hydrocarbons may be produced when decomposing polymers such as polyethylene, polypropylene, polystyrene, trioxane, or polyoxymethylene, which could be undesirable due to condensation in low-temperature applications. Thus, devices and systems of the present disclosure may include one or more filters, sieves, traps, condensers, or other similar components to selectively remove an identified undesirable species from the product gas(es). Such components can be provided in association with the compartment in which the product gas(es) is/are formed by decomposition of the polymer, or they can be provided in association with a separate compartment into which the one or more product gases flow after formation.

In some embodiments, it may be desirable to provide for further chemical processing of the one or more product gases. Particularly, it may be desirable to provide for subsequent chemical reaction of one or more product gases, e.g. gas production or gas reformation. In such embodiments, the devices and methods of the invention may employ a catalyst configured to facilitate such chemical processing of the one or more product gases. Such catalyst may be provided in any desired spatial arrangement (e.g. a fixed bed), and may be present either in the compartment in which the one or more product gases are formed (i.e. the compartment containing the polymer), or in a separate compartment configured to receive the one or more product gases.

In embodiments of the present disclosure, the polymer may be selected based on the identity of the gas or gases desired to be produced. By way of non-limiting example, where a gas desired to be produced is ethylene gas, polyethylene may be selected as the polymer. In some embodiments, the desired gas may be a secondary decomposition product, i.e. a gas that is produced by first thermally decomposing the polymer into an intermediate species and then further thermally or catalytically decomposing the intermediate species to the desired gas, and the polymer may be selected accordingly; by way of non-limiting example, where a desired gas is hydrogen gas, polyethylene may be selected as the polymer, and the gas generator device 100 may be configured to first decompose the polyethylene to ethylene gas and subsequently (due to, e.g., increased temperature or the presence of a catalyst) decompose the ethylene gas to hydrogen gas. Other polymers suitable for producing these or other product gases include polypropylene, polystyrene, trioxane, and polyoxymethylene.

In embodiments of the present disclosure, the polymer may be provided in any suitable physical form. By way of first non-limiting example, the polymer may be provided in a physical form comprising one or more pellets. By way of second non-limiting example, the polymer may be provided in a physical form comprising one or more sheets. By way of third non-limiting example, the polymer may be provided in a physical form comprising one or more tubes. By way of fourth non-limiting example, the polymer may be provided in a physical form comprising one or more rods. By way of fifth non-limiting example, the polymer may be provided in a physical form comprising one or more fibers. By way of sixth non-limiting example, the polymer may be provided in a physical form comprising one or more molded shapes or articles.

While the foregoing disclosure has generally focused on the production of gases in the context of inflating an inflatable article, it is to be expressly understood that the devices and methods of the disclosure are suitable to produce one or more product gases for any desired application. By way of first non-limiting example, the devices and methods of the disclosure may be used to fill or pressurize a cylinder, tank, or vessel, e.g. a storage cylinder or tank, with a desired gas. By way of second non-limiting example, the devices and methods of the disclosure may be used to produce a lifting gas to be used in, e.g., a buoyant vehicle or article such as a hot air balloon or a float. By way of third non-limiting example, the devices and methods of the disclosure may be used to produce a selected atmosphere within a volume, e.g. ethylene gas may be produced and used in a “ripening room” to accelerate the ripening of fruits and vegetables. These and other applications are within the scope of the present disclosure.

Several variations and modifications of the disclosure can be used. It would be possible to provide for some features of the disclosure without providing others.

The present disclosure, in various aspects, embodiments, and configurations, includes components, methods, processes, systems and/or apparatus substantially as depicted and described herein, including various aspects, embodiments, configurations, sub-combinations, and subsets thereof. Those of skill in the art will understand how to make and use the various aspects, embodiments, and configurations, after understanding the present disclosure. The present disclosure, in various aspects, embodiments, and configurations, includes providing devices and processes in the absence of items not depicted and/or described herein or in various aspects, embodiments, and configurations hereof, including in the absence of such items as may have been used in previous devices or processes, e.g., for improving performance, achieving ease and/or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more, aspects, embodiments, and configurations for the purpose of streamlining the disclosure. The features of the aspects, embodiments, and configurations of the disclosure may be combined in alternate aspects, embodiments, and configurations other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed aspect, embodiment, or configuration. Thus, the following claims are hereby incorporated into this Detailed Description, with each claim standing on its own as a separate preferred embodiment of the disclosure.

Moreover, though the description of the disclosure has included description of one or more aspects, embodiments, or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

1. A gas generator device, comprising:
a first compartment, containing a heat-generating composition;
a second compartment, containing a polymer; and
a separator in thermal contact with the first and second compartments, configured to transfer thermal energy generated in the first compartment by reaction of the heat-generating composition to the second compartment to thermally decompose the polymer to release at least one product gas.

2. The gas generator device of claim 1, wherein the heat-generating composition is a thermite composition, comprising a metal and a metal oxide.

3. The gas generator device of claim 2, wherein the metal oxide is selected from the group consisting essentially of vanadium (V) oxide, iron (III) oxide, iron (II,III) oxide, copper (II) oxide, copper (I) oxide, tin (IV) oxide, titanium dioxide, manganese dioxide, manganese (III) oxide, chromium (III) oxide, cobalt (II) oxide, silicon dioxide, nickel (II) oxide, silver oxide, molybdenum trioxide, lead (II,IV) oxide, bismuth (III) oxide, and mixtures thereof, and wherein the metal is selected from the group consisting of aluminum, magnesium, silicon, manganese, alloys of magnesium and aluminum, and mixtures thereof.

4. The gas generator device of claim 2, wherein the thermite composition comprises more than one metal, more than one metal oxide, or both.

5. The gas generator device of claim 1, wherein the polymer is selected from the group consisting of polyethylene, polypropylene, polystyrene, trioxane, polyoxymethylene, and combinations and mixtures thereof.

6. The gas generator device of claim 5, wherein the polymer comprises at least one of high-density polyethylene and low-density polyethylene.

7. The gas generator device of claim 1, wherein the at least one product gas comprises at least one of ethylene gas and hydrogen gas.

8. The gas generator device of claim 1, further comprising an igniter interconnected with the first compartment, configured to induce the reaction in the heat-generating composition upon initiation by one or more of a spark, heat, flame, and friction.

9. The gas generator device of claim 1, further comprising at least one gas transport channel in fluid communication with the second compartment, configured to direct flow of the product gas.

10. The gas generator device of claim 1, wherein the second compartment further contains a catalyst configured to promote the thermal decomposition of the polymer.

11. The gas generator device of claim 1, wherein the thermal decomposition of the polymer releases at least two product gases, and wherein the second compartment further contains a catalyst configured to control the thermal decomposition of the polymer to promote a desired production ratio between two or more of the at least two product gases.

12. The gas generator device of claim 1, further comprising a third compartment in fluid communication with the second compartment, configured to receive the at least one product gas from the second compartment and containing a catalyst configured to promote catalytic reforming of the at least one product gas.

13. The gas generator device of claim 1, configured to promote further decomposition of the at least one product gas to a secondary product gas by at least one of thermal decomposition and catalytic decomposition.

14. The gas generator device of claim 13, wherein the at least one product gas comprises ethylene gas and the secondary product gas comprises hydrogen gas.

15. The gas generator device of claim 1, configured to cool the at least one product gas.

16. The gas generator device of claim 15, further comprising a cooling compartment in fluid communication with the second compartment, configured to receive the at least one product gas from the second compartment and cool the at least one product gas.

17. The gas generator device of claim 1, further comprising a component configured to remove or separate a selected species from the at least one product gas.

18. The gas generator device of claim 17, wherein the component is selected from the group consisting of a condenser, a filter, a sieve, and a trap.

19. A gas generator device configured to release at least one product gas by thermal decomposition of a polymer, comprising:
a compartment, containing a heat-generating composition and a polymer; and
an igniter interconnected with the compartment, configured to induce a reaction in the heat-generating composition upon initiation by one or more of a spark, heat, flame, and friction,
wherein at least one of the following is true:
(i) the heat-generating composition and the polymer are physically located in a common chamber;
(ii) the heat-generating composition and the polymer are physically mixed together;
(iii) the heat-generating composition and the polymer are in direct physical contact;
(iv) the heat-generating composition and the polymer are spatially arranged proximate to each other to facilitate transfer of thermal energy generated by the reaction of the heat-generating composition to the polymer;
(v) the heat-generating composition is provided as a shaped or molded article comprising voids and the polymer occupies at least a portion of the voids; and
(vi) the polymer is provided as a shaped or molded article comprising voids and the heat-generating composition occupies at least a portion of the voids.

20. The gas generator device of claim 19, wherein the heat-generating composition is a thermite composition, comprising a metal and a metal oxide.

21. The gas generator device of claim 20, wherein the metal oxide is selected from the group consisting of vanadium (V) oxide, iron (III) oxide, iron (II,III) oxide, copper (II) oxide, copper (I) oxide, tin (IV) oxide, titanium dioxide, manganese dioxide, manganese (III) oxide, chromium (III) oxide, cobalt (II) oxide, silicon dioxide, nickel (II) oxide, silver oxide, molybdenum trioxide, lead (II,IV) oxide, bismuth (III) oxide, and mixtures thereof, and wherein the metal is selected from the group consisting of aluminum, magnesium, silicon, manganese, an alloy of magnesium and aluminum, and mixtures thereof.

22. The gas generator device of claim 20, wherein the thermite composition comprises more than one metal, more than one metal oxide, or both.

23. The gas generator device of claim 19, wherein the polymer is selected from the group consisting of polyethylene, polypropylene, polystyrene, trioxane, polyoxymethylene, and combinations and mixtures thereof.

24. The gas generator device of claim 23, wherein the polymer comprises at least one of high-density polyethylene and low-density polyethylene.

25. The gas generator device of claim 19, wherein the at least one product gas comprises at least one of ethylene gas and hydrogen gas.

26. The gas generator device of claim 19, further comprising at least one gas transport channel in fluid communication with the compartment, configured to direct flow of the product gas.

27. The gas generator device of claim 19, wherein the at least a portion of the polymer is provided as a pellet or rod having a coating of a heat-resistant material that moderates heat transfer to the polymer.

28. The gas generator device of claim 19, wherein the compartment further contains a catalyst configured to promote the thermal decomposition of the polymer.

29. The gas generator device of claim 28, wherein the catalyst is produced in situ as a byproduct of the reaction of the heat-generating composition.

30. The gas generator device of claim 19, wherein the thermal decomposition of the polymer releases at least two product gases, and wherein the compartment further contains a catalyst configured to promote a desired production ratio between two or more of the at least two product gases.

31. The gas generator device of claim 19, further comprising a second compartment in fluid communication with the first compartment, configured to receive the at least one product gas from the compartment and containing a catalyst configured to promote catalytic reforming of the at least one product gas.

32. The gas generator device of claim 19, configured to promote further decomposition of the at least one product gas to a secondary product gas by at least one of thermal decomposition, catalytic decomposition, and catalytic reformation.

33. The gas generator device of claim 32, wherein the at least one product gas comprises ethylene gas and the secondary product gas comprises hydrogen gas.

34. The gas generator device of claim 19, configured to cool the at least one product gas.

35. The gas generator device of claim 34, further comprising a cooling compartment in fluid communication with the compartment, configured to receive the at least one product gas from the compartment and cool the at least one product gas.

36. The gas generator device of claim 19, further comprising a component configured to remove or separate a selected species from the at least one product gas.

37. The gas generator device of claim 36, wherein the component is selected from the group consisting of a condenser, a filter, a sieve, and a trap.

38. A method for generating at least one product gas, comprising:
initiating reaction of a heat-generating composition to release thermal energy;
causing the transfer of at least some of the released thermal energy to a polymer; and
decomposing, with the thermal energy transferred to the polymer, at least some of the polymer to release the at least one product gas.

39. The method of claim 38, wherein the polymer is a polymer that can decompose to yield ethylene and the at least one product gas comprises ethylene.

40. The method of claim 39, wherein the polymer comprises polyethylene, wherein the heat-generating composition is a thermite composition comprising a metal and a metal oxide, wherein the metal oxide is selected from the group consisting essentially of vanadium (V) oxide, iron (III) oxide, iron (II,III) oxide, copper (II) oxide, copper (I) oxide, tin (IV) oxide, titanium dioxide, manganese dioxide, manganese (III) oxide, chromium (III) oxide, cobalt (II) oxide, silicon dioxide, nickel (II) oxide, silver oxide, molybdenum trioxide, lead (II,IV) oxide, bismuth (III) oxide, and mixtures thereof, and wherein the metal is selected from the group consisting of aluminum, magnesium, silicon, manganese, alloys of magnesium and aluminum, and mixtures thereof.

41. The method of claim 39, wherein the initiating step comprises contacting the heat-generating composition with an igniter initiated by one or more of a spark, heat, flame, and friction.

42. The method of claim 39, wherein thermal energy is transferred from the heat-generating composition to the polymer via a separator.

43. The method of claim 39, wherein the at least one product gas further comprises hydrogen gas.

44. The method of claim 39, further comprising cooling the at least one product gas.

45. The method of claim 38, further comprising inflating an inflatable article with the at least one product gas.

46. The method of claim 45, wherein the inflatable article is selected from the group consisting of a balloon and a float.
